(19) United States
(12) Patent Application Publication
(10) Pub. No.: US 2012/0156263 A1
(43) Pub. Date: Jun. 21, 2012

(54) Ursodeoxycholic Acid-Synthetic Hydrotalcite-Eudragit Hybrid, Pharmaceutical Composition Containing the Same and Method for Preparing the Same

(75) Inventors: Jin Ho Choy, Seoul (KR); Go Eun Choi, Seoul (KR); Myung Chul Park, Bucheon-city (KR); Hee Chang, Seoul (KR)

(73) Assignee: Daewoong Pharmaceutical Co., Ltd., Kyunggi-do (KR)

(21) Appl. No.: 13/392,770

(22) PCT Filed: Sep. 10, 2010
(86) PCT No.: PCT/KR2010/006192

§ 371 Date: Feb. 27, 2012

(30) Foreign Application Priority Data
Sep. 11, 2009 (KR) 10-2009-0086149

(51) Int. Cl. A61K 9/00; A61P 1/16; B29B 9/12; A61K 31/575

(52) U.S. Cl. 424/400; 514/182; 264/7

(57) ABSTRACT

The present invention relates to an ursodeoxycholic acid-synthetic hydrotalcite-Eudragit hybrid, a pharmaceutical composition containing the same and a method for preparing the same. The ursodeoxycholic acid-synthetic hydrotalcite-Eudragit hybrid according to the present invention is very useful as an active ingredient of a pharmaceutical composition because of its bitter-taste-blocking effect and improved body absorption rate with high solubility.

### TECHNICAL FIELD

The present invention relates to an ursodeoxycholic acid-synthetic hydrotalcite-Eudragit hybrid, a pharmaceutical composition containing the same and a method for preparing the same. More preferably, in the ursodeoxycholic acid-synthetic hydrotalcite-Eudragit hybrid according to the present invention, ursodeoxycholic acid is incorporated between the layers of synthetic hydrotalcite and Eudragit is coated on the hybrid.

### BACKGROUND ART

Ursodeoxycholic acid (UDCA) is one of the components comprised in bile which is produced in the liver. The capacity of the human liver to produce ursodeoxycholic acid is very small, and thus continual ingestion of ursodeoxycholic acid can be very helpful for liver health. More specifically, as disclosed in Korean Patent Publication No. 1997-0000042, ursodeoxycholic acid, silymarin, etc. are known as drugs for promoting regeneration of liver cells or assisting liver function, and a pharmaceutical composition for recovery from fatigue comprising ursodeoxycholic acid, red ginseng and vitamins is also disclosed. Korean Patent Publication No. 1997-0005178 discloses a pharmaceutical composition comprising tauroursodeoxycholic acid to show a recovery effect from fatigue.

However, because pure ursodeoxycholic acid has an intrinsic bitter taste, it causes a problem of bitter taste when a drug is not only orally administered but also refluxed due to reflux oesophagitis caused by oxidative stress from hypersecretion of acids in gastric ulcer or gastritis. Thus, it has been needed to block the bitter taste of ursodeoxycholic acid and thus more easily administer the drug for increasing drug-administration compliance.

### DISCLOSURE OF INVENTION

### Technical Problem

The object of the present invention is to provide an ursodeoxycholic acid-synthetic hydrotalcite-Eudragit hybrid, a pharmaceutical composition containing the same and a method for preparing the same.

### Solution to Problem

The present inventors performed intensive studies for developing a formulation by which ursodeoxycholic acid is administered more easily and drug-administration compliance is increased. As a result, they surprisingly found that a hybrid obtained by incorporating ursodeoxycholic acid between the layers of hydrotalcite, which is used as an antacid and a stomachic, and then coating with Eudragit, which is an enteric coating, blocks the bitter taste of ursodeoxycholic acid and simultaneously shows improvement of dissolution rate and high bioavailability; thus they completed the present invention.

Accordingly, to accomplish the above object the present invention provides an ursodeoxycholic acid-synthetic hydrotalcite-Eudragit hybrid represented by the following Formula 1 and a pharmaceutical composition comprising the same as an active ingredient:

[MgAl(OH)₆][C₂₄H₃₉O₄][C₉H₁₄O₃]x.y(H2O) [Formula 1]

wherein,

C₂₄H₃₉O₄ is an anionic form of ursodeoxycholic acid;

C₉H₁₄O₃ is Eudragit; and

x and y are a positive number above 0.

The present invention also provides a method for preparing an ursodeoxycholic acid-synthetic hydrotalcite-Eudragit hybrid comprising the steps of: (a) dissolving ursodeoxycholic acid in a weak basic aqueous solution to prepare an aqueous solution containing ursodeoxycholic acid; (b) mixing the aqueous solution containing ursodeoxycholic acid prepared in step (a) with an aqueous solution containing magnesium salt and aluminum salt, and then adding a basic aqueous solution to prepare a hybrid in which ursodeoxycholic acid is incorporated between the layers of synthetic hydrotalcite that is a co-precipitated product from said magnesium salt and aluminum salt; and (c) spray-drying Eudragit to said hybrid to prepare an Eudragit-coated hybrid.

The present invention also provides a method for preparing a pharmaceutical composition comprising the steps of: (a) dissolving ursodeoxycholic acid in a weak basic aqueous solution to prepare an aqueous solution containing ursodeoxycholic acid; (b) mixing the aqueous solution containing ursodeoxycholic acid prepared in step (a) with an aqueous solution containing magnesium salt and aluminum salt and then adding a basic aqueous solution to prepare a hybrid in which ursodeoxycholic acid is incorporated between the layers of synthetic hydrotalcite that is a co-precipitated product from said magnesium salt and aluminum salt; (c) spay-drying Eudragit to said hybrid to prepare an Eudragit-coated hybrid; and (d) formulating said coated hybrid to various formulations according to pharmaceutically acceptable formulas and processes.

### Advantageous Effects of Invention

According to the present invention, because the intrinsic bitter taste of ursodeoxycholic acid is blocked by the incorporation of ursodeoxycholic acid between the layers of synthetic hydrotalcite and the coating with Eudragit, adverse reaction at the time of oral administration is reduced, and ursodeoxycholic acid is selectively released in the intestines so as to increase pharmacological effects. The formulation comprising a hybrid according to the present invention shows increased effects because of the enhanced administration convenience and administration compliance

### BRIEF DESCRIPTION OF DRAWINGS

FIG. 1 is a graph showing the dissolution rate of pure ursodeoxycholic acid and the dissolution rate of ursodeoxycholic acid from ursodeoxycholic acid-synthetic hydrotalcite-Eudragit hybrid (a: pure ursodeoxycholic acid, b: ursodeoxycholic acid released from ursodeoxycholic acid-synthetic hydrotalcite-Eudragit hybrid).

FIG. 2 is a series of photographs for evaluating the dispersion stability of a pharmaceutical composition comprising an ursodeoxycholic acid-synthetic hydrotalcite-Eudragit hybrid at severe condition.

### MODE FOR THE INVENTION

The present invention is described in detail hereinafter.

According to one aspect, the present invention relates to an ursodeoxycholic acid-synthetic hydrotalcite-Eudragit hybrid represented by the following Formula 1 and a pharmaceutical composition comprising the same as an active ingredient:

[MgAl(OH)₆][C₂₄H₃₉O₄][C₉H₁₄O₃]x.y(H2O) [Formula 1]

wherein,

C₂₄H₃₉O₄ is an anionic form of ursodeoxycholic acid;

C₉H₁₄O₃ is Eudragit; and

x and y are a positive number above 0.

The term “hybrid” used herein refers to a form in which ursodeoxycholic acid is incorporated between the layers of synthetic hydrotalcite and bound by electrostatic attraction. In addition, it should be understood that the term “hybrid” as above also includes a form in which Eudragit is coated on the hybrid by electrostatic attraction.

In the present invention, “comprising A as an active ingredient” means that ingredient A is comprised to an extent that would to show medical effects of any positive influences such as improvement of liver function, prevention and treatment of liver diseases, recovery from fatigue, alleviation of fatigue symptoms, etc.

In the present invention, “ursodeoxycholic acid” represented by the following Formula 2 is one of the components comprised in bile which is produced in the liver, and because the capacity of the human liver to produce ursodeoxycholic acid is very small, and thus continual ingestion of ursodeoxycholic acid can be very helpful for liver health.

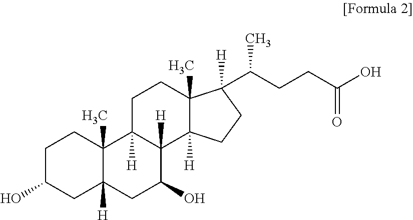

Hydrotalite generally represented by the following Formula 3 is a naturally existing basic, inorganic mineral, and is used as an antacid and protectant against gastric acid in an orally administrable pharmaceutical composition for treating gastric acidity increase, etc., and as a drug excipient to increase the powdery property of a solid formulation of the composition.

[Mg₆Al₂(OH)₁₆][CO₃].4(H₂O) [Formula 3]

Since hydrotalcite has a laminar structure consisting of inorganic lattice layers of magnesium-aluminum (ratio 3:1) and carbonic anion array between the layers, it is known that various anionic drug molecules can be introduced between the layers.

In the present invention, “synthetic hydrotalcite” refers to hydrotalcite obtained by synthesis and not hydrotalcite existing in nature, and can be prepared by the following preparation method according to the present invention. In comparison with the naturally existing hydrotalcite of the above Formula 3, the synthetic hydrotalcite of the present invention has a magnesium-aluminum ratio of 2:1 and does not comprise anion of carbonic acid. Such synthetic hydrotalcite is one of the ingredients comprised in the ursodeoxycholic acid-synthetic hydrotalcite-Eudragit hybrid of the above Formula 1.

In the present invention, Eudragit is a poly-methacrylate based copolymer used for enteric coating. Because Eudragit is dissolved only in the intestines of above pH 7, ursodeoxycholic acid, which is the active ingredient of the hybrid, can be selectively released from the hybrid so as to increase its pharmacological effects and minimize side effects. One example of Eudragit is Eudragit S 100 (Evonik Degussa GmbH).

Besides, the present composition may further comprise a dissolution aid. Examples of dissolution aids include various surfactants such as pharmaceutically acceptable anionic, cationic, nonionic or zwitterionic surfactants. More specifically, examples of surfactants include polyoxyethylene sorbitan fatty acid ester, sorbitan fatty acid ester, polyoxyethylene fatty acid ester and the like.

In the present invention, the above pharmaceutical composition may further comprise pharmaceutically acceptable additives. Examples of additives include, but are not limited thereto, excipient which is safe and used for increasing stability, specifically for increasing weight in solid formulation; diluent which is used for increasing volume in liquid formulation; disintegrant; binder which gives consistent adsorption, solidification, or the like to a mixture; lubricant which is used for providing slip and reducing friction; suspending agent which allows particles to be mixed well; surfactant; sweetener; preservative; flavor; thickener; pH regulator; wetting agent and mixture thereof. Such additives may be added according to a conventionally known formulation method.

A syrup formulation in which ingredients are homogeneously mixed may be prepared by properly mixing the appropriate amount of surfactant, sweetener, preservative, flavor, thickener, pH regulator, wetting agent and suspending agent within a pharmaceutically acceptable range without affecting the effects of the active ingredient. A syrup formulation having a proper liquid dispersity may be prepared by mixing proper carrier, excipient, diluent, or the like. For example, one or more biocompatible solid or liquid fillers (e.g., water, ethanol, propylene glycol, glycerin or mixture thereof), sweetener or flavor, pigment or dye, emulsifier and/or suspending agent (if necessary) may be mixed and included. Examples of such ingredients include, but are not limited thereto, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate and mineral oil.

In the present invention, the hybrid may be a hybrid in which said ursodeoxycholic acid is incorporated between the layers of synthetic hydrotalcite and Eudragit is coated. The coating may be carried out by spray-drying.

According to another aspect, the present invention relates to a method for preparing an ursodeoxycholic acid-synthetic hydrotalcite-Eudragit hybrid comprising the steps of: (a) dissolving ursodeoxycholic acid in a weak basic aqueous solution to prepare an aqueous solution containing ursodeoxycholic acid; (b) mixing the aqueous solution containing ursodeoxycholic acid prepared in step (a) with an aqueous solution containing magnesium salt and aluminum salt, and then adding a basic aqueous solution to prepare a hybrid in which ursodeoxycholic acid is incorporated between the layers of synthetic hydrotalcite that is a co-precipitated product from said magnesium salt and aluminum salt; and (c) spray-drying Eudragit to said hybrid to prepare an Eudragit-coated hybrid.

In the present invention, the ursodeoxycholic acid-synthetic hydrotalcite hybrid of the above steps (a) and (b) is preferably prepared by a co-precipitation method, and examples of solvent include, but are not limited thereto, distilled water, alcohol or mixture thereof. Alcohol is preferably ethanol. The examples of magnesium salt and aluminum salt include, but are not limited thereto, MgCl₂, Mg(NO₃)₃, Mg(CH₃COO)₂, AlCl₃, Al(NO₃)₃, Al(CH₃COO)₃ or hydrates thereof. In the present invention, the reaction concentration of magnesium salt and aluminum salt is, for example, 0.01 M to 5 M, and the use amount of ursodeoxycholic acid is, for example, about 0.1 to 10 molar ratio based on total moles of magnesium salt and aluminum salt. In the precipitation reaction, a base may be added to induce precipitation, if necessary. A suitable base is, for example, sodium hydroxide, potassium hydroxide, magnesium hydroxide, calcium hydroxide or ammonia. In the present invention, the pH of the reaction solution is preferably 8 to 11, more preferably 9 to 10, and the reaction temperature is preferably 0° C. to 100° C., more preferably 15° C. to 30° C. The reaction time is preferably more than 10 minutes. In addition, it is preferable to feed nitrogen or inert gas continuously during the reaction.

In the present invention, the coating of Eudragit in step (c) may be carried out by dispersing the hybrid in a solution containing Eudragit and then drying it. It is preferable to use spray-drying for good uniformity. Spray-drying is advantageous in that the drying is fast, and fine particles below 100 microns can be made.

In the present invention, the content of ursodeoxycholic acid in the ursodeoxycholic acid-synthetic hydrotalcite hybrid of the above step (b) and the ursodeoxycholic acid-synthetic hydrotalcite-Eudragit hybrid of the above step (c) is preferably 1 to 50% by weight.

According to still another aspect, the present invention relates to a method for preparing a pharmaceutical composition comprising the steps of: (a) dissolving ursodeoxycholic acid in a weak basic aqueous solution to prepare an aqueous solution containing ursodeoxycholic acid; (b) mixing the aqueous solution containing ursodeoxycholic acid prepared in step (a) with an aqueous solution containing magnesium salt and aluminum salt, and then adding a basic aqueous solution to prepare a hybrid in which ursodeoxycholic acid is incorporated between the layers of synthetic hydrotalcite that is a co-precipitated product from said magnesium salt and aluminum salt; (c) spray-drying Eudragit to said hybrid to prepare an Eudragit-coated hybrid; and (d) formulating said coated hybrid to various formulations according to a pharmaceutically acceptable formula and process.

The ursodeoxycholic acid-synthetic hydrotalcite-Eudragit hybrid according to the present invention may be prepared as a pharmaceutical composition of all pharmaceutical formulations such as syrup, powder, granule, capsule, tablet, suspension, chewable tablet, oral soluble film formulation, oral disintegrating tablet, liquid agent, dry syrup, etc. through various conventionally known formulation steps.

The preferable unit dose of pharmaceutical composition according to the present invention varies depending on various factors such as age, sex, etc. of the administration subject but is generally 10 to 500 mg, preferably 50 to 300 mg based on ursodeoxycholic acid.

Hereinafter, the present invention is explained in more detail with the following examples. However, it must be understood that the protection scope of the present invention is not limited to the examples.

### EXAMPLE 1

### Preparation of Ursodeoxycholic Acid-Synthetic Hydrotalcite-Eudragit Hybrid

1-1. Preparation of Ursodeoxycholic Acid-Synthetic Hydrotalcite Hybrid

MgCl₂.6H₂O (0.2 M) and AlCl₃.9H₂O (0.1 M) were dissolved in distilled and deionized water. Ursodeoxycholic acid (0.15 M) (Daewoong Pharmaceutical Co. Ltd., Korea) was dissolved in a weakly basic aqueous solution. Then two solutions were mixed and titrated to pH 9-10 with 1 M NaOH aqueous solution to precipitate and obtain ursodeoxycholic acid-synthetic hydrotalcite hybrid. The obtained hybrid was stirred at room temperature for 20 hours, and then unreacted salt was removed by filtering under reduced pressure and washing to obtain a hybrid wherein ursodeoxycholic acid is incorporated between the layers of synthetic hydrotalcite. The above process for preparing hybrid was carried out under nitrogen atmosphere to prevent the generation of carbonate ion (CO₃²⁻) by carbon dioxide in air.

1-2. Preparation of Ursodeoxycholic Acid-Synthetic Hydrotalcite-Eudragit Hybrid

The ursodeoxycholic acid-synthetic hydrotalcite hybrid prepared in the above step 1-1 was resuspended in a mixed solvent of methylene chloride and ethanol, and then Eudragit S 100 (Evonik Degussa GmbH, Germany) dissolved in ethanol was added thereto (ursodeoxycholic acid-synthetic hydrotalcite hybrid:Eudragit=1:1.5 weight ratio). The resulting solution was stirred and then spray-dried to obtain an ursodeoxycholic acid-synthetic hydrotalcite-Eudragit hybrid having a coating of Eudragit S 100.

### EXPERIMENTAL EXAMPLE 1

### Dissolution Test of Ursodeoxycholic Acid from Ursodeoxycholic Acid-Synthetic Hydrotalcite-Eudragit Hybrid

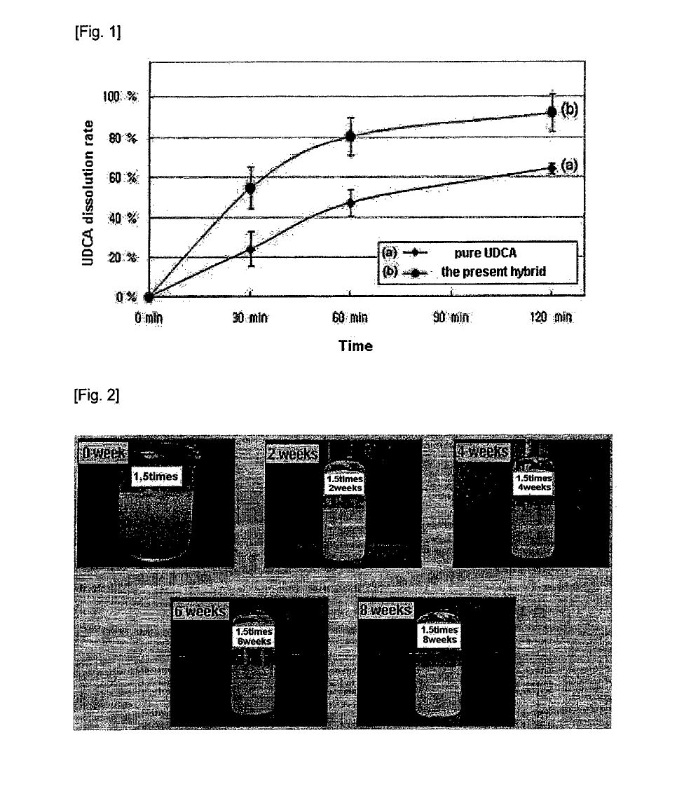

The dissolution test of the ursodeoxycholic acid-synthetic hydrotalcite-Eudragit hybrid prepared in the above Example 1 was carried out according to USP ursodiol tablet dissolution test (pH 8.0 buffer, 50 rpm, 37° C.). The results are represented in FIG. 1.

As can be seen from FIG. 1, the dissolution of ursodeoxycholic acid from ursodeoxycholic acid-synthetic hydrotalcite-Eudragit hybrid was 80% within 60 minutes and 90% within 120 minutes. Such a dissolution pattern is about 30% higher than that of pure ursodeoxycholic acid. Thus, it was confirmed that the present ursodeoxycholic acid-synthetic hydrotalcite-Eudragit hybrid would be very useful for increasing drug efficacy due to the increase of body absorption rate by selective release of ursodeoxycholic acid in the intestines.

### EXAMPLE 2

### Preparation of Pharmaceutical Composition Comprising Ursodeoxycholic Acid-Synthetic Hydrotalcite-Eudragit Hybrid

The pharmaceutical composition comprising an ursodeoxycholic acid-synthetic hydrotalcite-Eudragit hybrid as a main ingredient was prepared with ingredients as listed in the following Table 1.

TABLE 1

| Ingredient | Amount | |
|---|---|---|
| Ursodeoxycholic acid-synthetic hydrotalcite-Eudragit hybrid | 10 | g |
| Fructose | 1 | kg |
| Di-sorbitol | 0.3 | kg |
| Beta-cyclodextrin | 1 | g |
| Methyl paraoxybenzoate | 0.8 | g |
| Propyl paraoxybenzoate | 2 | g |
| Masking flavor | 27 | g |

Beta-cyclodextrin was dissolved in purified water, and then the ursodeoxycholic acid-synthetic hydrotalcite-Eudragit hybrid (main ingredient) and other ingredients shown in the above Table 1 were added thereto and dissolved. Purified water was further added to make a syrup formulation with final volume of 10 L according to a conventional method.

### COMPARATIVE EXAMPLE 1

### Preparation of Pharmaceutical Composition Comprising Pure Ursodeoxycholic Acid

The pharmaceutical composition was prepared by the same method as described in Example 1 except that pure ursodeoxycholic acid was used as the main ingredient.

### EXPERIMENTAL EXAMPLE 2

### Evaluation of Bitter-Taste-Blocking Effect of Pharmaceutical Composition Comprising Ursodeoxycholic Acid-Synthetic Hydrotalcite-Eudragit Hybrid

The pharmaceutical composition comprising ursodeoxycholic acid-synthetic hydrotalcite-Eudragit hybrid prepared in the above Example 2 was orally administered to 30 healthy adults, and then the level of bitter taste was evaluated at 5 minutes after administration. Evaluation results were divided into six (6) grades and are represented in the following Table 2. For comparison, the pharmaceutical composition comprising pure ursodeoxycholic acid prepared in Comparative Example 1 was also tested.

TABLE 2

| | Comparative | |
|---|---|---|
| Grade of bitter taste | Example 1 | Example 2 |
| 0: No bitter taste | 0 person | 30 persons |
| 1: Faint bitter taste | 0 person | 0 person |
| 2: Weak bitter taste | 0 person | 0 person |
| 3: Bitter taste | 0 person | 0 person |
| 4: Strong bitter taste but tolerable | 2 persons | 0 person |
| 5: Strong bitter taste and intolerable | 28 persons | 0 person |

As a result of sensory evaluation, it can be known that the pharmaceutical composition comprising ursodeoxycholic acid-synthetic hydrotalcite-Eudragit hybrid can reduce adverse reaction at the time of oral administration and increase administration convenience by successfully blocking the bitter taste of ursodeoxycholic acid.

### EXPERIMENTAL EXAMPLE 3

### Evaluation of Dispersion Stability of Pharmaceutical Composition Comprising Ursodeoxycholic Acid-Synthetic Hydrotalcite-Eudragit Hybrid

To evaluate the dispersion stability of ursodeoxycholic acid-synthetic hydrotalcite-Eudragit hybrid to temperature and humidity, it was tested at accelerated storage conditions as in the following Table 3 for 8 weeks. The results were initially evaluated and then every two weeks, and were represented in FIG. 2.

TABLE 3

| Package form | Brown glass bottle sealed with plastic cap |
|---|---|
| Storage temperature | 40 ± 1° C. |
| Storage humidity | 75 ± 5% RH |

As can be seen from FIG. 2, it was confirmed that the pharmaceutical composition comprising ursodeoxycholic acid-synthetic hydrotalcite-Eudragit hybrid according to the present invention maintained a dispersion state over the long term (8 weeks) even at accelerated conditions. Thus, the present pharmaceutical composition is expected to have an excellent practical use as a formulation which can increase therapeutic effects because of the improved administration convenience and administration compliance.

The pharmaceutical composition comprising ursodeoxycholic acid-synthetic hydrotalcite-Eudragit hybrid according to the present invention shows very good stability as well as good blocking effect against the bitter taste at sensory test. In addition, at dissolution test, it can be known that the present pharmaceutical composition selectively releases ursodeoxycholic acid in the intestines, and thus is very useful industrially.

Hereinbefore, the present invention and its embodiments have been described in detail. However, it would be evident for a person skilled in the art that the scope of the present invention is not intended to be limited to the particular embodiments, and various modifications and variations can be made without departing from the spirit of the present invention.

1. An ursodeoxycholic acid-synthetic hydrotalcite-Eudragit hybrid represented by the following Formula 1:
[MgAl(OH)₆][C₂₄H₃₉O₄][C₉H₁₄O₃]x.y(H2O) [Formula 1]
wherein,
C₂₄H₃₉O₄ is an anionic form of ursodeoxycholic acid;
C₉H₁₄O₃ is Eudragit; and
x and y are each independently a positive number greater than 0.

2. The ursodeoxycholic acid-synthetic hydrotalcite-Eudragit hybrid according to claim 1, wherein ursodeoxycholic acid is incorporated between the layers of synthetic hydrotalcite and Eudragit is coated on the hybrid.

3. A pharmaceutical composition comprising the ursodeoxycholic acid-synthetic hydrotalcite-Eudragit hybrid according to claim 1 as an active ingredient.

4. The pharmaceutical composition according to claim 3 wherein the content of ursodeoxycholic acid in the ursodeoxycholic acid-synthetic hydrotalcite-Eudragit hybrid is from 1 to 50% by weight.

5. The pharmaceutical composition according to claim 3 which further comprises a pharmaceutically acceptable additive.

6. The pharmaceutical composition according to claim 5, wherein the pharmaceutically acceptable additive is selected from the group consisting of diluents, disintegrants, binders, lubricants, surfactants, sweeteners, preservatives, flavors, thickeners, pH regulators, wetting agents and mixtures thereof.

7. A method for preparing an ursodeoxycholic acid-synthetic hydrotalcite-Eudragit hybrid comprising the steps of:
(a) dissolving ursodeoxycholic acid in a weak basic aqueous solution to prepare an aqueous solution containing ursodeoxycholic acid;
(b) mixing the aqueous solution containing ursodeoxycholic acid prepared in step (a) with an aqueous solution containing magnesium salt and aluminum salt and then adding a basic aqueous solution to prepare a hybrid in which ursodeoxycholic acid is incorporated between the layers of synthetic hydrotalcite that is a co-precipitated product from said magnesium salt and aluminum salt; and
(c) spray-drying Eudragit to said hybrid to prepare an Eudragit-coated hybrid.
